(19) United States
(12) Patent Application Publication
(10) Pub. No.: US 2017/0143287 A1
(43) Pub. Date: May 25, 2017

(54) SELF-ADJUSTING X-RAY IMAGING METHOD FOR STEREOTACTIC BIOPSY, X-RAY SOURCE CONTROL DEVICE, AND STEREOTACTIC BIOPSY MACHINE

(71) Applicant: SIEMENS HEALTHCARE GMBH, Erlanger (DE)

(72) Inventor: TOM WEIDNER, ERLANGEN (DE)

(21) Appl. No.: 15/359,756

(22) Filed: Nov. 23, 2016

(30) Foreign Application Priority Data
Nov. 23, 2015 (DE) 10 2015 223 074.0

(51) Int. Cl. A61B 6/00; A61B 6/12; A61B 10/02

(52) U.S. Cl. CPC A61B 6/4452; A61B 6/5205; A61B 6/502; A61B 10/0233; A61B 6/54; A61B 6/12

(57) ABSTRACT

Stereo projections of breast tissue to be examined are dynamically generated with the aid of an x-ray projection machine. An x-ray source of the x-ray projection machine is moved to a start position. Thereupon, an x-ray projection recording is started at the start position and the x-ray projection recording is performed while the x-ray source moves to a finish position. The position of the x-ray focus is updated during the x-ray projection recording so that a change in position of the x-ray focus is compensated for at least in part, preferably in full. The x-ray projection recording is terminated when the finish position is reached. A method for ascertaining a position of breast tissue to be examined and an automated method for planning a biopsy are described. An x-ray source control device and an automated stereotactic biopsy machine are described.

### CROSS-REFERENCE TO RELATED APPLICATION

This application claims the priority, under 35 U.S.C. §119, of German patent application DE 10 2015 223 074.9, filed Nov. 23, 2015; the prior application is herewith incorporated by reference in its entirety.

### BACKGROUND OF THE INVENTION

### Field of the Invention

The invention relates to a method for dynamically generating stereo projections of breast tissue to be examined. Moreover, the invention relates to a method for ascertaining a position of breast tissue to be examined. Furthermore, the invention also relates to an automated method for planning a biopsy. Moreover, the invention relates to an x-ray source control device. Moreover, the invention relates to a stereotactic biopsy machine.

Mammography continues to play an important role for the early detection of breast cancer. In conventional mammography, an x-ray recording of the female breast is created. The x-ray radiation used in the process is soft radiation with an energy of approximately 25 to 35 keV. For the purpose of detecting the x-ray radiation, use is made of direct digital detectors and indirect digital detectors to capture the emitted x-ray radiation. Direct digital detectors convert the x-ray radiation directly into an electrical signal. By contrast, indirect digital detectors first of all convert the x-ray radiation into visible light, which is subsequently converted into an electrical signal. The x-ray recordings are observed on a special mammography findings station which comprises one or two grayscale monitors, by means of which the x-ray images are presented pictorially.

In order to be able to clarify findings, the biopsy method is often performed in addition to the mammography. In the process, tissue of the examined breast considered to be conspicuous on the basis of a mammography recording is removed with a type of hollow needle. Since this process is quite uncomfortable for patients, it is desirable to design the examination duration to be as short as possible.

In order to prepare an automated stereotactic biopsy, a so-called scout image recording is performed first in order to set a region of the breast tissue in which the tissue to be removed is situated. Subsequently, two x-ray projection recordings of the set region are performed from different directions, for example from ±15°, relative to the normal on the detector plane. The position of the tissue to be removed is ascertained on the basis of the recordings. A recording with two projection recordings from different directions is necessary for ascertaining the z-position of the tissue to be removed. The z-position should be understood to mean the coordinate in the direction of the z-axis of a coordinate system, the z-axis of which extends in the direction of the surface normal of the detector surface. After the position of the tissue to be removed has been ascertained, the correct needle position in the xy-plane, i.e., in the plane parallel to the detection surface, is set on the basis of this information and the needle is displaced to this position. Thereafter, the needle penetrates the breast tissue and is guided to just in front of the z-position of the breast tissue to be removed. Once the needle has been inserted, verification images are now recorded once again in a stereo projection, i.e., from different directions, in order to ascertain whether the needle position is correct or whether a correction is necessary. Subsequently, the biopsy is carried out, i.e. the needle is guided to the exact z-position of the tissue to be removed and the tissue to be removed is extracted from the breast region. After removal, verification images are once again recorded in order to ascertain whether the entire target tissue has in fact been removed from the breast. Subsequently, the hollow needle is removed again from the breast to be examined and the removed tissue may now be used for diagnostic purposes.

Thus, overall, stereo projection images of the tissue region to be examined are created three times. As already mentioned, the x-ray source needs to be displaced between two different positions to this end. The second x-ray image recording may only be started once the x-ray source is situated at the new position. As already mentioned, this procedure requires a great amount of time.

### SUMMARY OF THE INVENTION

It is accordingly an object of the invention to provide a method and a device, which overcomes the above-mentioned disadvantages of the heretofore-known devices and methods of this general type and which provides for a faster process for obtaining stereo projection data with sufficient precision.

With the foregoing and other objects in view there is provided, in accordance with the invention, a method of dynamically generating stereo projections of breast tissue to be examined with an x-ray projection machine. The method comprises the following steps:

moving an x-ray source of the x-ray projection machine to a start position and starting an x-ray projection recording at the start position;

moving the x-ray source towards a finish position and performing the x-ray projection recording while the x-ray source is moving towards the finish position;

updating a position of an x-ray focus of the x-ray source during the x-ray projection recording in dependence on a calculated change in a position of the x-ray source to thereby compensate, at least in part, for a change in position of the x-ray focus on account of the movement of the x-ray source to the finish position; and

terminating the x-ray projection recording when the finish position of the x-ray source is reached.

The objects of the invention are achieved with the above-summarized method for dynamically generating stereo projections of breast tissue to be examined, with a method for ascertaining a position of breast tissue to be examined, as claimed, an automated method for planning a biopsy, as claimed, an x-ray source control device, as claimed, and a stereotactic biopsy machine, as claimed.

In the method according to the invention for dynamically generating stereo projections of breast tissue, to be examined, with the aid of an x-ray projection machine, a position of an image recording region is preferably ascertained on the basis of scout image data in a breast to be examined. As already mentioned, a scout image recording is a simple two-dimensional orientation recording with a lower resolution. An image recording region of interest may, for example, contain a lesion or another conspicuous tissue structure which is intended to be analyzed in a subsequent diagnosis method.

Subsequently, a start position of an x-ray focus is advantageously ascertained on the basis of the established position of the image recording region. An x-ray focus should be understood to mean the focus of the electron beam of the x-ray tube of an x-ray source. It also corresponds to the center of an x-ray beam emanating from the x-ray source. An x-ray tube usually comprises an electrode system comprising a cathode and an anode. Electrons are accelerated from the cathode to the anode and trigger x-ray radiation upon incidence on the anode, which x-ray radiation may be used to image an object to be examined.

In biopsy units, the image recording region is usually specified by the positioning and the working region of the biopsy unit, and so ascertaining the position of the image recording region and the start position of the x-ray focus may also be dispensed with when applying the method in conjunction with such biopsy units.

Thereupon, an x-ray source of the x-ray projection machine is displaced to a start position. In the process, the x-ray source is moved to a position from which an x-ray projection recording should be started. Thereafter, an x-ray projection recording is started at the start position and the x-ray projection recording is performed while the x-ray source moves to a finish position. In contrast to a conventional x-ray projection recording, the recording in the method according to the invention is therefore already performed during a movement of the x-ray source. Hence, the x-ray projection recording may already be started when the x-ray source has not yet quite reached its actual finish position. In this way, at least some of the recording time is saved.

During the x-ray projection recording, the position of the x-ray focus of the x-ray source is updated in such a way that a change in position of the x-ray focus is compensated for at least in part, preferably in full. An improvement in the image quality of the x-ray projections is achieved by virtue of the movement of the x-ray source being at least partly compensated. Since there consequently is a reduction in the movement of the x-ray beam on the detector surface, said movement causing an imaging aberration, the image quality may be improved with, at the same time, a shortened recording time. Finally, the x-ray projection recording is terminated when the target position of the x-ray source is reached.

In the method according to the invention for ascertaining a position of breast tissue to be examined, the method according to the invention for dynamically generating stereo projections of breast tissue to be examined is performed from a first start position to a first finish position. Subsequently, the method for dynamically generating stereo projections of breast tissue to be examined is carried out from a second start position to a second finish position. Finally, the position of the tissue to be examined is ascertained on the basis of the two captured x-ray projections. Thus, two x-ray projection recordings from different positions are required in order to be able to ascertain the three-dimensional position of a tissue region to be examined. On account of the compensation of the movement of the x-ray source by deflecting or updating the x-ray focus, it is possible to record the two x-ray projection recordings while the x-ray source is moving.

In the automated method for planning a biopsy according to the invention, a scout image recording is initially performed in order to set a region of the breast tissue in which the biopsy tissue is situated. Subsequently, the method according to the invention for ascertaining a position of breast tissue to be examined is carried out in order to obtain preliminary information for a position of a biopsy needle. Then, the exact position of the biopsy tissue is ascertained on the basis of the x-ray projection data captured in the method for ascertaining a position of breast tissue to be examined. Furthermore, a position of a biopsy needle in the breast tissue is now set on the basis of the ascertained position of the biopsy tissue.

Moreover, the position of the biopsy needle is preferably checked within the scope of this method after the biopsy needle was inserted into the breast tissue by using the method according to the invention for ascertaining a position of breast tissue to be examined. The described steps are preferably carried out in an automated manner such that there is no need for operating staff to intervene and the quality of the biopsy is independent of the personal skill of the operating staff.

The x-ray source control device according to the invention comprises an image information evaluation unit, a tissue position ascertainment unit and a computer unit. Image information of a scout image is evaluated with the aid of the image information evaluation unit to the extent that regions which contain tissue to be examined in more detail are identified. The tissue position ascertainment unit is configured to estimate the position of the identified regions on the basis of the available image information. However, as already mentioned, these estimates may only be carried out incompletely since no three-dimensional position of the tissue region of interest is ascertainable on the basis of the scout image. The computer unit serves to ascertain a position of an x-ray focus of an x-ray source depending on a start position of the x-ray source, the approximately estimated position of the tissue to be examined and the recording time after the start of the x-ray projection recording.

The automated stereotactic biopsy machine according to the invention comprises a biopsy apparatus with a controllable biopsy needle, preferably a hollow needle, an x-ray projection device for recording two-dimensional x-ray projections from a plurality of directions, the x-ray source control device according to the invention and a biopsy position ascertainment unit. The biopsy position ascertainment unit is configured to ascertain a three-dimensional position of the tissue to be removed within the scope of the biopsy on the basis of the x-ray projections captured by the x-ray projection device. Furthermore, the automated stereotactic biopsy machine according to the invention comprises an actuation device configured to perform a biopsy in an automated manner on the basis of the ascertained tissue position. By way of example, a biopsy needle is controlled with the aid of the actuation device in order to take a tissue sample at the ascertained biopsy position.

The essential components of the biopsy machine according to the invention may, to a large extent, be embodied in the form of software components. In particular, this relates to parts of the x-ray source control device such as e.g. the image information evaluation unit and the computer unit, as well as the biopsy position ascertainment unit. However, in principle, these components may also be realized in the form of software-assisted hardware, e.g., FPGAs (field-programmable gate arrays) or the like, particularly if particularly fast calculations are important. Likewise, the required interfaces may be embodied as software interfaces, for example if all that is important is a transfer of data from other software components. However, they may also be embodied as interfaces designed in terms of hardware, which are actuated by suitable software.

An implementation largely in terms of software is advantageous in that previously used stereotactic biopsy machines may also be retrofitted in a simple manner by means of a software update in order to operate in the inventive manner. In this respect, the object is also achieved by a corresponding computer program product comprising a computer program, which is directly loadable into a memory unit of a control device of a biopsy machine according to the invention, comprising program sections to run all steps of the method according to the invention when the computer program is run by the control device. By way of example, the control device may comprise the x-ray source control device according to the invention. Additionally, it may also comprise further control units for carrying out the planning of a biopsy.

In addition to the computer program, such a computer program product may optionally comprise additional components such as, say, documentation and/or additional components, hardware components as well, such as e.g. hardware keys (dongles, etc.) for using the software.

A computer-readable medium, for example a memory stick, a hard disk drive or any other transportable or securely installed data medium, on which the program sections of the computer program which may be read and executed by a computer unit of the control device are stored, may serve for the purposes of transporting to the memory device of the control device of the biopsy machine, for example the x-ray source control device, and/or for storing in the memory device. By way of example, to this end the computer unit may have one or more cooperating microprocessors or the like.

The dependent claims and the subsequent description in each case contain particularly advantageous configurations and developments of the invention. Here, in particular, the claims of one claim category may also be developed in a manner analogous to the dependent claims of another claim category. Moreover, the various features of different exemplary embodiments and claims may also be combined to form new exemplary embodiments within the scope of the invention.

In one configuration of the method according to the invention for dynamically generating stereo projections of breast tissue, to be examined, with the aid of an x-ray projection machine, the position of the x-ray focus is updated by controlling the deflection of the electron beam of the x-ray source. By way of example, the electron beam may be influenced in terms of the profile thereof with the aid of electromagnets which are arranged about a trajectory of the electron beam. Preferably, the electron beam is deflected counter to a movement direction of the x-ray source such that there is no change, or at least less variation, in the position of the x-ray focus than in the case without a deflection of the electron beam. An improved image quality is achieved in this manner.

In accordance with a preferred configuration of the method according to the invention for dynamically generating stereo projections of breast tissue, to be examined, with the aid of an x-ray projection machine, the deflection of the electron beam is controlled dependent on the speed of the movement of the x-ray source and the elapsed x-ray projection recording time. The current position of the x-ray source or the change in position thereof may be ascertained from the speed with which the x-ray source is moving and the time since the start of the x-ray projection recording. This corresponds to necessary deflection of the electron beam to compensate the change in position of the x-ray source. Using this procedure, it is possible to exactly match the deflection of the electron beam of the x-ray tube of the x-ray source to the movement or the current position thereof in space.

Preferably, a two-dimensional image data record is reconstructed on the basis of projection data of the x-ray projection recording in the method according to the invention for dynamically generating stereo projections of breast tissue, to be examined, with the aid of an x-ray projection machine. By way of example, x-ray projection data may be corrected in terms of noise effects or stray-radiation effects before an image data record is generated therefrom, and so the quality of the image data record is improved.

In accordance with a particularly advantageous configuration of the method according to the invention for ascertaining a position of breast tissue to be examined, the first start position has a predetermined angle position relative to the surface normal of the detection surface of an x-ray detector used for the projection recordings and the second start position has an angle position with the same magnitude but reversed sign. Furthermore, the first finish position has a predetermined angle position relative to the surface normal of the detection surface of the x-ray detector used for the projection recordings and the second finish position has an angle position with the same magnitude but reversed sign. By way of example, the respective start position and the respective finish position may be grouped about a mean value. By way of example, if the generation of the two projection recordings at the positions P and −P is desired, the start positions may lie at P−ΔP and −P+ΔP and the finish positions may lie at P+ΔP and −P−ΔP. By way of example, an x-ray source drives from a central position 0 to the position P−ΔP, starts the first x-ray projection recording and continues the latter until it arrives at the position P+ΔP. Subsequently, the x-ray source drives to the position −P+ΔP and starts the second x-ray projection recording, which it completes at the position −P−ΔP. By way of example, P may have an angle value 15°; ΔP may for example, without being restricted thereto, have a value of 0.5° or a value between 0.5° and 1°.

Within the scope of a preferred variant of the method according to the invention for ascertaining a position of breast tissue to be examined, the tissue to be examined comprises biopsy tissue.

After carrying out a biopsy, the success of the biopsy is preferably checked by using the method according to the invention for ascertaining a position of breast tissue to be examined. Here, whether the tissue of interest was successfully or completely removed from the breast is ascertained on the basis of the captured stereo projections or the image data reconstructed therefrom.

In a particularly practical variant of the method according to the invention for planning a biopsy, the success of the biopsy is checked, after carrying out the biopsy, using the method according to the invention for ascertaining a position of breast tissue to be examined. Such monitoring is preferably carried out directly after removing a biopsy sample while the biopsy needle is still present in the breast of a patient. If it is determined that the entire tissue to be extracted has not been removed, the residual tissue may be removed without a renewed repositioning process or merely with small positional corrections of the biopsy needle.

Other features which are considered as characteristic for the invention are set forth in the appended claims.

Although the invention is illustrated and described herein as embodied in self-adjusting x-ray imaging method for the stereotactic biopsy and related devices, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic view of an arrangement for carrying out a stereotactic biopsy;

FIG. 2 is a schematic illustration of the movement of an x-ray focus during an x-ray recording with a moving x-ray source;

FIG. 3 is a schematic illustration of the behavior of the x-ray focus when applying the method in accordance with the invention;

FIG. 4 is a schematic illustration of a control device of an x-ray source; and

FIG. 5 is a flowchart which illustrates a method for dynamically generating stereo projections of breast tissue to be examined in accordance with one exemplary embodiment of the invention.

### DETAILED DESCRIPTION OF THE INVENTION

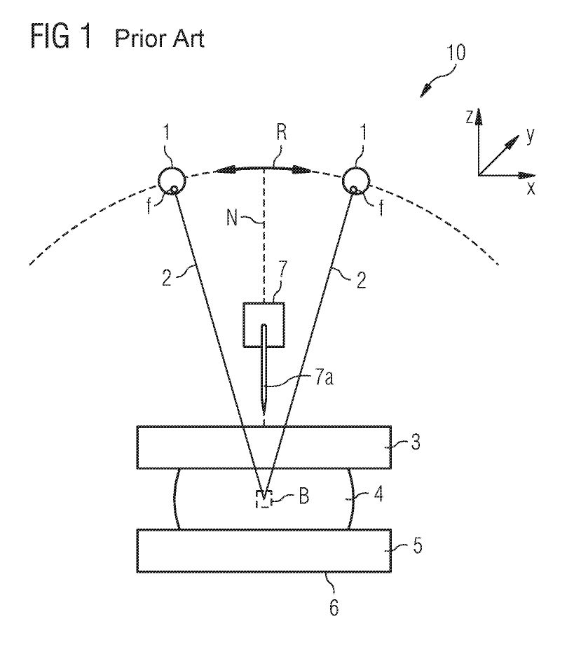

Referring now to the figures of the drawing in detail and first, particularly, to FIG. 1 thereof, there is seen an arrangement 10 for stereotactic biopsy. The arrangement comprises an x-ray source 1, which emits x-ray radiation 2 in the direction of a breast 4. FIG. 1 only plots the chief ray of the x-ray beam 2. However, the x-ray beam 2 may cover a broad region of the object to be imaged, i.e. the breast 4. The breast 4 lies on an object table 5 and is pressed against the object table 5 by a compression plate 3. In this way, the thickness of the breast is reduced in the z-direction, i.e. in the direction of the surface normal N of the object table 5. Reducing the thickness of the object irradiated by the x-ray radiation is accompanied by a reduction in the stray radiation, contributing to an improved image quality. Some of the x-ray radiation incident on the breast 4 is absorbed. The rest of the x-ray radiation incident on the breast 4 is transmitted and captured by an image sensor or x-ray detector 6. The x-ray source 1 is variable in terms of its position. That is to say, the x-ray source 1 may be positioned at various angles relative to the surface normal N of the object table 5. To this end, the x-ray source 1 is displaceable in the R direction. Depending on the position of the x-ray source 1, x-ray projections of the breast 4 may be recorded from different angles.

As already mentioned, a plurality of x-ray projection recordings of a set region of the breast 4 are carried out from different directions, for example from ±15°, relative to the normal N on the object table 5 or the detector plane for the purposes of planning and monitoring an automated stereotactic biopsy. Then, the position of the tissue to be removed is ascertained on the basis of these recordings. This process is elucidated in FIG. 1 by depicting the x-ray source 1 in two different positions, one to the right of the centrally arranged surface normal N and one to the left of the surface normal N. FIG. 1 shows that the focus f of the x-ray source 1 moves with the x-ray source 1 when the latter is moved in the R direction. Then, the exact position of the tissue to be removed is ascertained on the basis of two x-ray projections of a region B to be examined, captured from different directions. FIG. 1 moreover shows an automated biopsy apparatus 7 comprising a hollow needle 7a which, on the basis of the position information of the tissue to be removed is controlled exactly to the position of said tissue in order to remove the biopsy tissue there. As already mentioned, the x-ray source 1 needs to be displaced to different positions for recording the two x-ray projections, harboring a certain amount of time outlay. Since the x-ray projection recordings must be carried out with a hollow needle already inserted into the breast tissue, this means that the patient must suffer the overall procedure for a longer period of time.

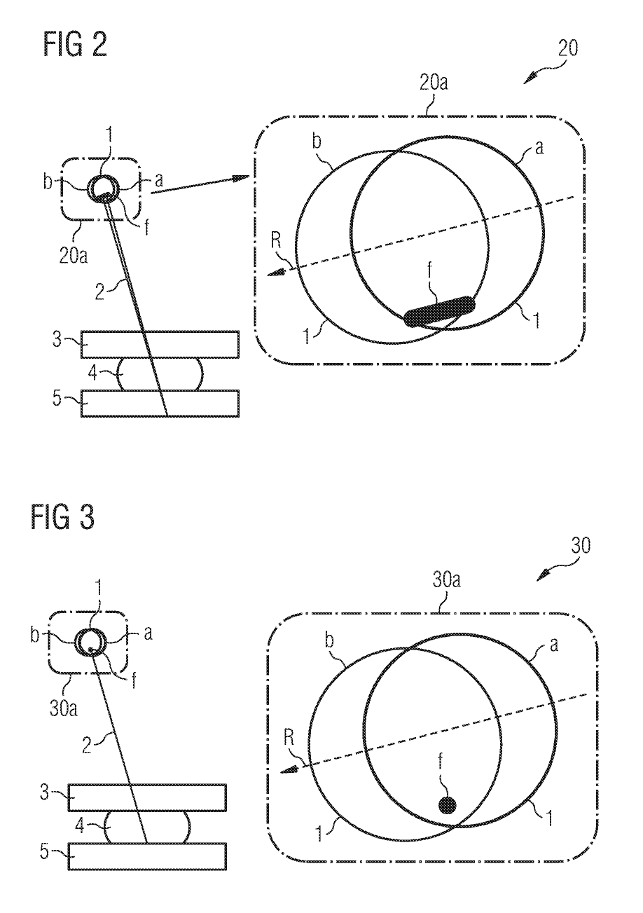

FIG. 2 shows a schematic illustration 20 which elucidates a procedure for recording the x-ray projections, with the x-ray projections in this procedure already being started during the drive to a position of the x-ray source 1. By way of example, if a projection recording is intended to be performed at an angle of +15°, the projection recording is already started at an angle of 14.5° relative to the normal N, while the x-ray source is still moving to the actual recording position at +15°. The x-ray source 1 is also moved slightly beyond the angle of +15° to a finish position at 15.5° until the recording is completed and the x-ray source comes to rest. The movement of the x-ray source 1 during the x-ray recording from a start position a to a finish position b is once again depicted in a magnified manner to the right in FIG. 2 in a partial drawing 20a. As can be identified therein, the focus f of the x-ray source 1 moves together with the x-ray source 1. However, an imaging aberration arises in the x-ray projection on account of this movement since different regions of the breast tissue to be imaged are now imaged on one point of the detector surface 6 due to the shift of the x-ray focus f.

FIG. 3 shows a schematic illustration 30 which shows a procedure in accordance with one exemplary embodiment of the invention which solves the problem of the imaging aberration in the case of a moving x-ray source 1. Here, the x-ray focus f is moved counter to the movement direction of the x-ray source 1 while an x-ray projection of the breast tissue to be imaged is recorded such that there is no change in the position of the x-ray focus f despite a moving x-ray source 1. What this achieves is that the x-ray beam 2 of the x-ray source 1 is always incident on the detector surface 6 from the same direction when recording an x-ray projection, despite the x-ray source 1 moving in the direction R, and so a point in the tissue to be imaged is always imaged on the same point of the detector surface 6 during the whole recording time. These circumstances are depicted in a magnified manner in a right-hand partial drawing 30a. While the x-ray source 1 moves from a position a in the direction of a position b, the x-ray focus f remains in a reference system at rest, i.e. it remains at the same position in a reference system of the arrangement 30. By contrast, in the moving reference system of the x-ray source 1 itself, the x-ray focus f moves to the right. Such a change in the relative position of the x-ray focus f relative to the x-ray source 1 is achieved by deflection of the electron beam of the x-ray source 1 counter to the movement direction R of the x-ray source 1.

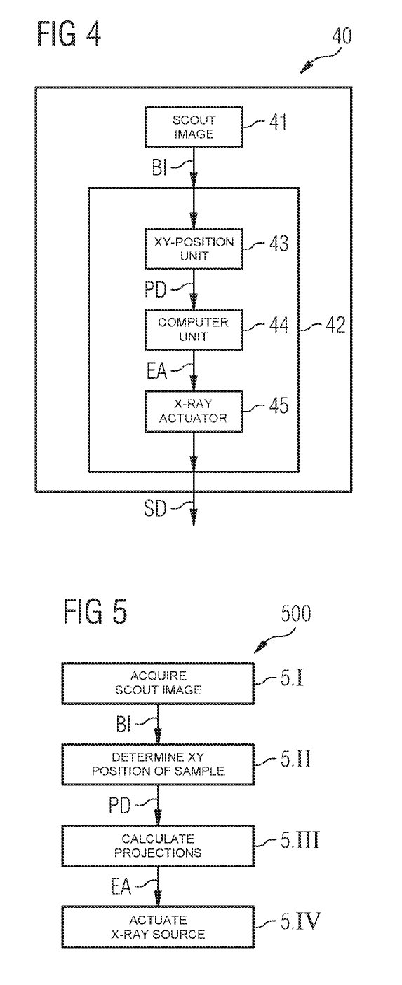

FIG. 4 shows a control device 40 of an x-ray source. The control device 40 comprises a scout image data evaluation unit 41, by means of which a scout image with the image information BI may be evaluated. By way of example, the scout image was already recorded as a low-resolution two-dimensional x-ray image in the direction of the surface normal of the detector plane. Lesions which should subsequently be subjected to a biopsy can be identified in the scout image. This image information BI is transferred to an x-ray source control device 42 which comprises a tissue position ascertainment unit 43, a computer unit 44 and an x-ray source actuation unit 45. With the aid of the tissue position ascertainment unit 43, an xy-position of the tissue to be examined is ascertained on the basis of the image information BI of the scout image. That is to say, a position of the tissue to be removed is ascertained in a plane parallel to the detector surface. The computer unit 44 serves now to ascertain a required electron beam deflection EA in the x-ray source 1 for the purposes of recording a plurality of x-ray projections from different directions on the basis of the position data PD ascertained on the basis of the scout image. By way of example, an x-ray deflection for a start position of the x-ray source, at which the x-ray source starts with recording an x-ray projection, and for a finish position, at which the recording of an x-ray projection is terminated, are ascertained. Furthermore, the deflection of the x-ray beam is ascertained during the x-ray projection recording, said deflection depending on the duration of the recording or the speed of the x-ray source with which the latter moves from the start position a to the finish position b. The ascertained data are forwarded to the x-ray source actuation unit 45 which controls the recording of the x-ray projections from different directions on the basis of this data and, in the process, transfers control data SD to the x-ray source 1 and to actuators, by way of which the movement of the x-ray source is controlled.

FIG. 5 shows a flowchart 500 elucidating a method for dynamically generating stereo projections of breast tissue, to be examined, with the aid of an x-ray projection machine in accordance with one exemplary embodiment of the invention. In step 5.1, image information BI is initially ascertained on the basis of the scout image, i.e. tissue regions from which a tissue sample is intended to be taken are identified. In step 5.11, position data in relation to the positions of the identified tissue regions are ascertained. That is to say, there is ascertainment of where a tissue sample to be taken is situated. By way of example, the xy-coordinates of this tissue region are ascertained on the basis of the captured image information BI. Thus, the scout data or the overview image are used to identify the tissue region of interest.

Subsequently, parameters for a plurality of the subsequent projection recordings are calculated in a step 5.111. By way of example, an electron beam deflection EA of an electron beam of an x-ray source, which should be used for recording a stereo projection, is established. Finally, there is an actuation of the x-ray source and of an x-ray detector of an x-ray recording system in step 5.IV for the purposes of recording a plurality of 2D projections from different directions. Here, the x-ray projection recordings already take place during the drive to a recording position of the x-ray source, i.e. with a moving x-ray source. For the purposes of compensating a movement of the x-ray focus with the moving x-ray source, an electron beam of the x-ray source is now deflected on the basis of the ascertained deflection data EA of the electron beam of the x-ray source in such a way that the movement of the x-ray source is compensated and the x-ray focus does not move. Only some of the breast tissue is scanned with the x-ray beam or the x-ray field of the x-ray source when recording the stereo projection. That is to say, the relevant region is restricted by the size of the biopsy window, i.e. the operating region of the biopsy attachment. The width of the x-ray field, in particular, is restricted by the operating region or the biopsy window. The operating region in turn is restricted by the arrangement of the compression plates, or the compression plate and the object table. Since the x-ray beam is planar, an entire volume is therefore irradiated at all times. The height of the region to be irradiated is known on the basis of the compression thickness of the breast. The z-coordinate of the object of interest is only calculated from the information from the two stereo recordings.

In closing, reference is once again had to the fact that the methods and apparatuses described above represent merely preferred exemplary embodiments of the invention and that the invention may be varied by a person skilled in the art without departing from the scope of the invention as predetermined by the claims. For the sake of completeness, reference is also made to the fact that the use of the indefinite article “a” or “an” does not preclude the relevant features from also being able to be present multiple times. Likewise, the term “unit” does not preclude the latter from consisting of a plurality of components which, where necessary, may also be spatially distributed.

The following is a summary list of reference numerals and the corresponding structure used in the above description of the invention:
1 X-ray source
2 X-ray beam
3 Compression plate
4 Breast
5 Object table
6 Image sensor/detector/detector surface
7 Biopsy apparatus
7a Biopsy needle/hollow needle
10 Arrangement for stereotactic biopsy
20 Illustration
20a Partial drawing
30 Illustration
30a Partial drawing
40 Control device
41 Scout image evaluation unit
42 X-ray source control device
43 Tissue position ascertainment unit
44 Computer unit
45 X-ray source actuation unit
a Start position
b Finish position
BI Image information
EA Electron beam deflection
f Focus of the x-ray source
N Surface normal
PD Position data
R Movement direction of the x-ray source
SD Control data

1. A method of dynamically generating stereo projections of breast tissue to be examined with an x-ray projection machine, the method comprising the following steps:
moving an x-ray source of the x-ray projection machine to a start position;
starting an x-ray projection recording at the start position;
moving the x-ray source towards a finish position and performing the x-ray projection recording while the x-ray source moves towards the finish position;
updating a position of an x-ray focus of the x-ray source during the x-ray projection recording in dependence on a calculated change in a position of the x-ray source to thereby compensate, at least in part, for a change in position of the x-ray focus on account of the movement of the x-ray source to the finish position; and
terminating the x-ray projection recording when the finish position of the x-ray source is reached.

2. The method according to claim 1, which comprises updating the x-ray focus to fully compensate for the change in the x-ray focus on account of the movement of the x-ray source.

3. The method according to claim 1, wherein the step of updating the position of the x-ray focus comprises controlling a deflection of the electron beam of the x-ray source.

4. The method according to claim 3, which comprises controlling the deflection of the electron beam in dependence on a speed of a movement of the x-ray source and an elapsed x-ray projection recording time.

5. The method according to claim 1, which comprises reconstructing a two-dimensional image data record on a basis of the x-ray projection recording.

6. A method of ascertaining a position of breast tissue to be examined, the method comprising the following steps:
performing the method according to claim 1 from a first start position to a first finish position;
subsequently performing the method according to claim 1 from a second start position to a second finish position;
ascertaining the position of the tissue to be examined on a basis of the two x-ray projection recordings thus obtained.

7. The method according to claim 6, wherein:
the first start position has a predetermined angle position relative to a surface normal of a detection surface of an x-ray detector used for the projection recordings and the second start position has an angle position with a same magnitude but reversed sign; and
the first finish position has a predetermined angle position relative to the surface normal of the detection surface and the second target position has an angle position with the same magnitude but reversed sign.

8. The method according to claim 6, wherein the tissue to be examined comprises biopsy tissue.

9. An automated method for planning a biopsy, the method comprising the following steps:
recording a scout image in order to set a region of the breast tissue in which biopsy tissue is situated;
performing the method according to claim 1 from a first start position to a first finish position;
subsequently performing the method according to claim 1 from a second start position to a second finish position;
ascertaining an exact position of the biopsy tissue on the basis of the captured x-ray projection recordings;
setting a position of a biopsy needle in the breast tissue based on the ascertained position of the biopsy tissue.

10. The method according to claim 9, which comprises monitoring a success of a biopsy after performing the biopsy by applying the method according to claim 6.

11. An x-ray source control device, comprising:
an image information evaluation unit for evaluating image information of a scout image;
a tissue position ascertainment unit configured for ascertaining from the image information a position of tissue to be examined;
a computer unit for ascertaining a position of an x-ray focus of an x-ray source in dependence on a start position of the x-ray source, the position of the tissue to be examined and a recording time after a start of an x-ray projection recording;
an actuation unit for updating the position of the x-ray focus of the x-ray source during an x-ray projection recording depending on a calculated change in position of the x-ray source to thereby compensate, at least in part, for a change in the position of the x-ray focus on account of the movement of the x-ray source to a finish position.

12. The control device according to claim 11, wherein said actuation unit is configured to update the x-ray focus to fully compensate for the change in the x-ray focus on account of the movement of the x-ray source.

13. An automated stereotactic biopsy machine, comprising:
a biopsy apparatus including a controllable biopsy needle;
an x-ray projection device for recording two-dimensional x-ray projections from a plurality of directions;
an x-ray source control device including:
an image information evaluation unit for evaluating image information of a scout image;
a tissue position ascertainment unit configured for ascertaining from the image information a position of tissue to be examined;
a computer unit for ascertaining a position of an x-ray focus of an x-ray source of said x-ray projection device in dependence on a start position of the x-ray source, the position of the tissue to be examined and a recording time after a start of an x-ray projection recording;
an actuation unit for updating the position of the x-ray focus of the x-ray source during an x-ray projection recording depending on a calculated change in position of the x-ray source to thereby compensate, at least in part, for a change in the position of the x-ray focus on account of the movement of the x-ray source to a finish position;
a biopsy position ascertainment unit for ascertaining a position of the tissue to be removed; and
an actuation device configured to perform a biopsy in an automated manner on a basis of the tissue position ascertained by said biopsy position ascertainment unit.

14. A computer program product, comprising a computer program, which is directly loadable into a memory unit of a control device of a biopsy machine according to claim 11, the computer program including program code configured to execute the steps according to the method of claim 1 when the computer program is run in the control device.

15. A computer readable medium having program sections stored thereon in non-transitory form which may be read and run by a control device of an automated stereotactic biopsy machine according to claim 11, to carry out the steps of the method according to claim 1 when the program sections are run by the control device.
